(19) United States
(12) Patent Application Publication
(10) Pub. No.: US 2010/0112713 A1
(43) Pub. Date: May 6, 2010

(54) Methods For Detecting Colorectal Diseases And Disorders

(75) Inventors: Robert S. Chapkin, College Station, TX (US); Laurie A. Davidson, College Station, TX (US); Joanne R. Lupton, College Station, TX (US); Edward R. Dougherty, JR., College Station, TX (US)

Correspondence Address: Peter G. Carroll;MEDLEN & CARROLL, LLP, Suite 350, 101 Howard Street, San Francisco, CA, 94105, US

(73) Assignee: The Texas A&M University System

(21) Appl. No.: 12/612,262

(22) Filed: Nov. 4, 2009

Related U.S. Application Data
(60) Provisional application No. 61/111,553, filed on Nov. 5, 2008.
(60) Provisional application No. 61/138,737, filed on Dec. 18, 2008.

(51) Int. Cl. G01N 33/48

(52) U.S. Cl. 436/94

(57) ABSTRACT

The present invention relates to methods and compositions for the detection of biomarkers associated with colorectal diseases and disorders. In preferred embodiments, said colorectal disease is colorectal cancer. In some embodiments, the invention relates to the detection of said biomarkers using non-invasive methods. In further embodiments, the invention relates to the isolation and evaluation of biomarkers residing in feces from a subject at risk for or exhibiting symptoms associated with a colorectal disease or disorder. In still further embodiments, said biomarkers include exfoliated colonocytes. In additional embodiments, mRNA transcripts isolated from said colonocytes and associated with said colorectal diseases and disorders are quantified.

### STATEMENT OF GOVERNMENT SUPPORT

This invention was made in part with government support under grant number S06-039, from the National Institutes of Health. As such, the United States government has certain rights to the invention.

### FIELD OF THE INVENTION

The present invention relates to methods and compositions for the detection of biomarkers associated with colorectal diseases and disorders. In preferred embodiments, said colorectal disease is colorectal cancer. In some embodiments, the invention relates to the detection of said biomarkers using non-invasive methods. In further embodiments, the invention relates to the isolation and evaluation of biomarkers residing in feces from a subject at risk for or exhibiting symptoms associated with a colorectal disease or disorder. In still further embodiments, said biomarkers include exfoliated colonocytes. In additional embodiments, messenger RNA (mRNA) transcripts isolated from said colonocytes and associated with said colorectal diseases and disorders are quantified.

### BACKGROUND OF THE INVENTION

Diseases and disorders of the colon and rectum, collectively referred to as the colorectal region, affect millions of people worldwide. One of the most recognizable diseases, colorectal cancer, is among the most common forms of cancer and a leading cause of cancer-related death in the Western world. Current methods for detecting colorectal cancer and pre-cancerous lesions and polyps are based largely on the use of invasive, tube-based cameras known as colonoscopes or sigmoidoscopes. The use of such devices is often a source of anxiety and extreme discomfort for a patient. Therefore, the development and implementation of non-invasive methods and assays for detecting biomedical indicators or biomarkers associated with colorectal cancer holds great appeal. However, current non-invasive methods lack both the necessary sensitivity of the aforementioned invasive techniques and the capacity for detecting alterations in the expression of genes associated with colorectal cancer. Thus, there is a need for the development of non-invasive methods for determining colorectal diseases and disorders that further allows for the examination of a patient's colonic gene expression profile.

### SUMMARY OF THE INVENTION

The present invention relates to methods and compositions for the detection of biomarkers associated with colorectal diseases and disorders. In preferred embodiments, said colorectal disease is colorectal cancer. In some embodiments, the invention relates to the detection of said biomarkers using non-invasive methods. In further embodiments, the invention relates to the isolation and evaluation of biomarkers residing in feces from a subject at risk for or exhibiting symptoms associated with a colorectal disease or disorder. In still further embodiments, said biomarkers include exfoliated colonocytes. In additional embodiments, mRNA transcripts isolated from said colonocytes and associated with said colorectal diseases and disorders are quantified.

In some embodiments, the invention relates to a method of detecting a biomarker associated with a colorectal disease or disorder comprising a) obtaining a fecal sample from a subject exhibiting symptoms associated with or at risk (e.g. at risk because of prior adenomas, at risk because of insulin resistance, at risk because of a history of adenomatous polyps, etc.) for said colorectal disease or disorder, b) isolating at least one biomarker from said fecal sample, and c) quantifying said biomarker. In further embodiments, said colorectal disease or disorder is selected from the group consisting of colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome. In still further embodiments, said fecal sample is obtained within two hours of excretion from said subject. In additional embodiments, said subject is a mammal. In some embodiments, said biomarker is messenger RNA. In further embodiments, said biomarker is associated with at least one gene. In still further embodiments, said gene is selected from the group consisting of ACADS, ADAM9, ALOX5, ALOX12B, ATOH1, AXIN2, BAX, BCL, BCL2L12, BECN, CEAL1, CDC42, CSPG2, CSPG4, CXCL-1, EGF, EGFR, F11R, FABP1, FOX, FOXD2, FOXD4L1, FOXL1, FOXL2, FOXP1, FOXP3, FOXD2, FOXO3A, GST-M4, GUCA2A, HMGCL, HOXA1, HOXA11, HOXB2, HOXB3, HOXD10, HSPA12B, ICAM1 (CD54), IGF2, IGFR-1, ITGB4BP, KAI1, KIT, MAPK11, MCM2, MUC5AC, NOX1, NPAT, OGG1, PCNA, PHB, PIK3R1, PIK3C2G, PLCG1, PLCG2, PLCD3, PLCD4, POLG, PRKACB, PTK2B, PTK2, SDC1, SPARC, TGFB2, TGFβ, TGM4, TIMP3, TNF, TNFRSF10B, UCP-3, WNT1, WNT3, Wnt3A, and Wnt5A.

In some embodiments, the invention relates to a method of measuring biomarker associated with a colorectal disease or disorder comprising a) obtaining a first fecal sample from a subject on a first diet, b) isolating mRNA from said fecal sample, c) determining a first mRNA profile, d) changing the diet of said subject to a second diet, f) obtaining a second fecal sample from a subject on said second diet, g) isolating mRNA from said fecal sample, h) determining a second mRNA profile, and j) comparing said first and second mRNA profiles. In further embodiments, said second mRNA profile indicates a reduced risk for developing adenomas. In still further embodiments, said second diet consists of consuming legumes. It is not intended that the present invention be limited by the precise nature of the diets employed. In one embodiment, a seven-day menu cycle is contemplated for the second diet with a standard set of legumes of the Phaseolus vulgaris species, such as, navy beans, pinto beans, and kidney beans in order to limit nutrient and phytochemical differences in the seven-day diet cycle. In further embodiments, the second diet contains at least 200 grams of legumes per day, more preferably approximately 250 grams of legumes per day. In still further embodiments, said second diet may be modified to provide other high glycemic index (GI) foods in the control or first diet such that the GI of the control or first diet has a GI of approximately 70 compared to a GI of 30 in the legume diet. In still further embodiments, said first diet and said second diet are controlled such that a constant level of energy available from dietary fat is maintained. In additional embodiments, the energy percentage of said dietary fat energy is at least 30%, more preferably between 32 and 33%. A further embodiment of the present invention is the use of a high legume, low glycemic index diet with a total dietary fiber intake of approximately 40 grams per day. In further embodiments, the invention relates to a corresponding high glycemic index diet comprising approximately 20 grams of total dietary fiber per day. A further embodiment of the present invention relates to the maintenance of the protein level of both the high glycemic index diet and the low glycemic index diet. In preferred embodiments, the energy percentage available from said protein level is at least 15%, preferably approximately 18%. It is further contemplated that said protein level is maintained through incorporation of protein sources including but in no way limited to red meat, fish and poultry.

In some embodiments, the present invention relates to a legume enriched, low glycemic index (GI), high fermentable fiber diet for reducing the risk of or symptoms associated with colorectal diseases and disorders in a subject. In further embodiments, said subject exhibits at least one risk factor. In still further embodiments, said risk factor includes but is in no way limited to insulin resistance and adenomatous polyps. In still further embodiments, at least one gene associated with a colorectal disease or disorder, and preferably at least two genes, are analyzed using the methods of the present invention. In additional embodiments, said gene or genes are analyzed for identifying subjects at risk for or exhibiting symptoms associated with risk factors including but not limited to adenomatous polyps and insulin resistance.

In some embodiments, the invention relates to a method of detecting a biomarker associated with a colorectal disease or disorder comprising a) obtaining a fecal sample from a subject exhibiting symptoms associated with or at risk (e.g. at risk because of prior adenomas, at risk because of insulin resistance, at risk because of a history of adenomatous polyps, etc.) for said colorectal disease or disorder, b) isolating at least one colonocyte from said fecal sample; c) further isolating at least one biomarker from said colonocyte, and d) quantifying said biomarker. In further embodiments, said colorectal disease or disorder is selected from the group consisting of colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome. In still further embodiments, said fecal sample is obtained within two hours of excretion from said subject. In additional embodiments, said subject is a mammal. In some embodiments, said biomarker is messenger RNA.

In some embodiments, the invention relates to a method of measuring biomarker associated with a colorectal disease or disorder comprising a) obtaining a first fecal sample from a subject on a first diet, b) isolating colonocytes from said first fecal sample; c) isolating mRNA from said colonocytes fecal samples; d) determining a first mRNA profile, e) changing the diet of said subject to a second diet, f) obtaining a second fecal sample from a subject on said second diet, g) isolating colonocytes from said second fecal sample; h) isolating mRNA from said colonocytes fecal samples; i) determining a second mRNA profile, and j) comparing said first and second mRNA profiles. In further embodiments, said second mRNA profile indicates a reduced risk for developing adenomas. In still further embodiments, said second diet consists of consuming only legumes.

### BRIEF DESCRIPTION OF THE DRAWINGS

For a more complete understanding of the features and advantages of the present invention, reference is now made to the detailed description of the invention along with the accompanying figures.

FIG. 1 shows a schematic overview of the experimental design as described in Example 1.

FIG. 2 shows the LDA classification (+IR, +Polyps)/class 0 (depicted as ◯), versus (−IR, −Polyps)/class 1 (Δ), at bl1 as described in Example 1. The concept of intrinsically multivariate predictive (IMP) genes is shown where expression profiles of a group of genes predict the phenotype. Results represent a linear classification of (+IR, +Polyps) subjects (◯) versus (−IR, −Polyps) subjects (Δ) at BL1. UCP2 and HOXA3 were used as individual one-feature sets (A and B) as compared with both genes together as a two-feature set (C). The bolstered error is 0.2784, 0.4882, and 0.1415 for (A), (B), and (C), respectively.

FIG. 3 shows the LDA classification (+IR, +Polyps)/class 0 (depicted as ◯), versus (−IR, −Polyps)/class 1 (Δ), at bl1 as described in Example 1. Effective classification of clinical phenotype or diet. (A), linear (LDA) classification of (+IR, +Polyps) subjects (◯) versus (−IR, −Polyps) subjects (Δ) at BL1; (B) linear (LDA) classification of (−IR, −Polyps) subjects on the control diet (◯) versus (−IR, −Polyps) subjects on the legume diet (Δ) using the crossover design and combining the microarrays from samples collected at the end of the two diet periods DP1 and DP2.

FIG. 4 shows the LDA classification (−IR, −Polyps, Control diet)/class 0 (depicted as ◯), versus (−IR, −Polyps, Legume diet)/class 1 (Δ) as described in Example 1. Potential design problems and importance of the experimental design factors IR and history of adenomas. (A) increased error in the LDA classification of (+IR, +Polyps) subjects (◯) versus (−IR, −Polyps) subjects (Δ) when both baselines BL1 and BL2 were included. (B) (+Polyps) subjects (◯) versus (−Polyps) subjects (Δ) at baselines BL1 and BL2. (C) (+IR) subjects (◯) versus (−IR) subjects (Δ) at all time points.

FIG. 5 shows the Housekeeping gene preparation. Two normalization issues were addressed. First, there was a large number of low-quality spots and second, while the microarray intensities showed no aberrant trend up to a certain point in time (relative to when microarray was performed), after a certain point there was a somewhat linear decline in intensity. Data points (blue dots) in FIG. 5 show the average values of the 18 housekeeping genes across microarrays, ordered from earliest to latest with respect to the time of processing. Common good probes (2,584) across all 86 microarrays were identified. A good probe is defined as having, at most, two low measures across all 86 microarrays. Using a list of 575 housekeeping genes (16), 18 genes were identified from the 2,584 probes found in the previous step. Subsequently, the raw intensity of each of the 18 housekeeping genes was quantified, and those with missing values were excluded. As a result, there were a total of 18 housekeeping genes used for normalization in Example 1. Arrays were grouped across time and the average values of 18 housekeeping genes were calculated in FIG. 5.

Table I shows the classification groups, sample sizes and number of common genes in the set A²₁∩B as described in Example 1. BL1 and BL2 indicate the base lines 1 or 2, +IR and −IR indicate present or absent insulin resistance, and +Polyps and −Polyps indicate presence or absence of polyps.

Table II shows the (+IR, +Polyps) data versus (−IR, −Polyps) data and BL1 as provided for in Example 1. Pair-wise or triplet-wise LDA classifiers are included when they rank higher than 20^{th} in both lists. ε_{bolstered} denotes the bolstered re-substitution error for the respective classifier; Δε_{bolstered} denotes the largest increase in error for the feature set relative to all of its subsets and ε_{resub} denotes the re-substitution as described in Example 1. Shows the classification of (+IR, +Polyps) subjects versus (−IR, −Polyps) subjects at BL1. Single-gene, pair-wise, and triplet-wise LDA classifiers are shown. ε_{bolstered} denotes the bolstered resubstitution error for the respective classifier; Δε_{bolstered} denotes the largest decrease in error for the feature set relative to all of its subsets.

Table III shows the (−IR, −Polyps) on control versus (−IR, −Polyps) on legume diet as provided for in Example 1. Pair-wise or triplet-wise LDA classifiers are included when they rank higher than 30^{th} in both lists. ε_{bolstered} denotes the bolstered re-substitution error for the respective classifier; Δε_{bolstered} denotes the largest increase in error for the feature set relative to all of its subsets and ε_{resub} denotes the re-substitution as described in Example 1. Shows the classification of (−IR, −Polyps) subjects on control diet versus (−IR, −Polyps) subjects on the legume diet. Single-gene, pair-wise, and triplet-wise LDA classifiers are shown. Refer to Table II for legend details.

Table IV shows the overall structure of the microarray data set.

Table V shows the Final classifier gene list.

Table VI A^{k}ⱼ∩B represents the number of genes that are common between the set B of established colonic biomarkers and the spots A^{k}ⱼ on the microarray set that passed quality threshold set by the parameters k and j. The value k=1.5 is the default value for the CodeLink image processing software, and j represents the number of accepted low (L) spots for a gene across all of the microarrays in the experiment.

Table VII shows the classification groups, sample size and number of common genes in each data set. BL1, baseline 1; BL2, baseline 2; +IR and IR indicate presence or absence of insulin resistance, respectively. +Polyps and −polyps indicate the presence or absence of polyps, respectively.

Table VIII shows Relative exfoliated cell gene expression levels in (+IR, +Polyps) vs (−IR, −Polyps) subjects at baseline 1 (BL1). Fold change represents the relative expression level in (+IR, +Polyps) subjects divided by (−IR, −Polyps) subjects for individual genes described in Table 1. p-values were computed using t-tests applied to the normalized data.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “colorectal disease” and “colorectal disorder” refer to diseases and disorders of the colon, and rectum. While not limiting the scope of the invention in any way, colorectal diseases and disorders include but are in no way limited to colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome.

As used herein, “colorectal cancer”, also known as “colon cancer”, “large rectal cancer” and “anal cancer,” is a disease that originates from the epithelial cells lining the gastrointestinal tract. The disease is often characterized by the cancerous growths residing in the colon and/or rectum. Symptoms associated with colorectal cancer include but are in no way limited to change in bowel habits, change in the appearance of stool including but not limited to bloody stool, rectal bleeding, stool with mucus, and/or black tar-like stool, bowel obstruction, the presence of an abdominal tumor, unexplained weight loss, jaundice, abdominal pain, anemia and blood clots.

A “colonocyte” refers to an epithelial cell that lines the mammalian colon.

As used herein, a “biomarker” is a substance used as an indicator of a biomedical state. While not limiting the scope of the present invention in any way, it is often a characteristic that is objectively measured and evaluated as an indicator of normal biomedical processes, pathogenic processes, or pharmacologic responses to a therapeutic intervention. A biomarker includes but is in no way limited to a nucleic acid sequence, peptide, protein, chemical modifier, chemical inhibitor, biomedical fluid or biomedical excrement. In preferred embodiments, the present invention relates to the detection and analysis of biomarkers associated with colorectal diseases and disorders. In even more preferred embodiments, said biomarker is messenger RNA. Examples of biomarkers associated with the detection of said colorectal diseases and disorders include but are in no way limited to biomarkers associated with ALOX12B (arachidonate 12-lipoxygenase), APC2 (adenomatous polyposis coli 2), Axin2 (conductin), BAD (bcl-2 antagonist of cell death), BECN1 (beclin 1), CA5B (carbonic anhydrase 5), CDC42 (G25K GTP-binding protein), CDK4 (cyclin-dependent kinase 4), CD44 (CD44 antigen), CSPG4 (chondroitin sulphate proteoglycan 4), CXCL-1 (chemokine CXC motif (GRO-alpha)), DAPK1 (death-associated protein kinase), EGF (epidermal growth factor), EGFR (epidermal growth factor receptor), FOXL1 (forkhead box protein L1), FOXL2 (forkhead box protein L2), FOXO1A (forkhead box protein O1A), FOXP3 (forkhead box protein P3), FOXP4 (forkhead box protein P4), FOXD2 (forkhead box protein D2), FOXO3A (forkhead box protein 3A), GST-M4 (glutathione S-transferase), GUCA2A (guanylate cyclase activator 2A), HOXA3 (homeobox gene A3), HOXB3 (homeobox gene B3), HOXC6 (homeobox gene C6), HOXD10 (homeobox gene D10), HSPA12B (heat shock protein A12B), ICAM1 (intracellular adhesion molecule 1 (CD54)), ID2 (inhibitor of DNA binding 2), IGF2 (insulin-like growth factor 2), IGFR-1 (insulin-like growth factor receptor 1), ITGB4BP (integrin beta 4 binding protein), KAI1 (CD82 tumor suppressor gene), KIT (proto-oncogen tyrosine-protein kinase), LEF-1 (lymphoid enhancer binding factor/T cell factor transcription factor), MAPK11 (mitogen activated protein kinase 11/p38 beta), MCM2 (minichromosome maintenance deficient 2), MUC5AC (secreted gel forming mucin 5AC), NOS3 (nitric oxide synthase 3), NOX1 (NADPH oxidase 1), NPAT (ataxia telangiectasia locus), OGG1 (8-oxoguanine DNA glycosylase), PCNA (proliferating cell nuclear antigen), PHB (prohibitin), PIK3R1 (phosphatidylinositol 3-kinase regulatory subunit p85 alpha), PIK3C2G (phosphoinositide 3-kinase, class 2, gamma polypeptide), PLCG2 (phosphatidylinositol-specific phospholipase gamma 2), PLCD4 (phospholipase C delta 4), POLG (DNA polymerase gamma), PRKACB (protein kinase, cyclic AMP-dependent, catalytic subunit beta), PTK2 (protein tyrosine kinase 2), SDC1 (syndecan 1), SFRP5 (secreted frizzled-related protein 5), SPARC, TGFβ (transforming growth factor beta 3), TNF (tumor necrosis factor), TNFRSF10B (tumor necrosis factor super family member 10B), TP53 (tumor suppressor protein p53), UCP-2 (uncoupling protein 2), UCP-3 (uncoupling protein 3), WNT1 (Wingless-type MMTV integration site family, member 1), Wnt3A (wingless-type MMTV integration site family member 3A), Wnt5A (wingless-type MMTV integration site family member 5A), YWHAZ (14-3-3 zeta).

As used herein, “energy percentage” is the percentage of energy, i.e. calories, derived from a macronutrient, including but in no way limited to carbohydrates, proteins and fats consumed by a subject.

As used herein, the terms “prevent” and “preventing” include the prevention of the recurrence, spread or onset of a disease or disorder. It is not intended that the present invention be limited to complete prevention. In some embodiments, the onset is delayed, or the severity of the disease or disorder is reduced.

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

“Subject” refers to any mammal, preferably a human patient, laboratory animal, livestock, or domestic pet.

### DETAILED DESCRIPTION OF THE INVENTION

The present invention relates to methods and compositions for the detection of biomarkers associated with colorectal diseases and disorders. In preferred embodiments, said colorectal disease is colorectal cancer. In some embodiments, the invention relates to the detection of said biomarkers using non-invasive methods. In further embodiments, the invention relates to the isolation and evaluation of biomarkers residing in feces from a subject at risk for or exhibiting symptoms associated with a colorectal disease or disorder. In still further embodiments, said biomarkers include exfoliated colonocytes. In additional embodiments, mRNA transcripts isolated from said colonocytes and associated with said colorectal diseases and disorders are quantified.

In preferred embodiments, the present invention relates to methods for the detection of colorectal diseases and disorders such as colorectal cancer. Early detection of colorectal cancer can greatly increase the prognosis for a subject exhibiting symptoms associated with the disease, thus it is desirable to have accurate screening methods and assays. Consistent with this goal, the adoption of non-invasive methodology designed to reduce anxiety over colorectal cancer screening and improve overall acceptance of the screening process would be highly desirable. Unfortunately, current non-invasive detection methods lack sensitivity and are incapable of detecting alterations in gene expression. This current limitation is significant because changes in gene expression can modulate the regulatory mechanisms that either promote or protect a subject against colorectal diseases and disorders such as colorectal cancer. Thus, the present invention utilizes a novel, non-invasive methodology based on the analysis of fecal or stool samples, which contain intact sloughed colon cells, in order to quantify colorectal disease and disorder relevant gene expression profiles.

Colon cancer is one of the leading causes of cancer-related deaths in the United States. Early detection is one of the proven strategies resulting in a higher cure rate (Rutter, 2006). Unfortunately, the currently adopted screening procedures for early detection are often invasive, e.g. colonoscopy, and discomfort associated with such procedures generally leads to resistance toward the screening process. Thus, adoption of noninvasive methodology designed to reduce anxiety over colorectal cancer screening and improve overall acceptance of the screening process would be highly desirable. See U.S. Pat. No. 6,258,541, hereby incorporated by reference.

Approximately one-sixth to one-third of normal adult colonic epithelial cells are shed daily as provided for in Potten (1979) Biochimica et Biophysica Acta 560, 281-299, incorporated herein by reference. The present invention provides for novel, non-invasive methodologies utilizing feces, which contain exfoliated colonocytes, in order to quantify colonic mRNAs as provided for in Davidson et al. (1995) Cancer Epidemiology Biomarkers and Prevention 4, 643-647; Davidson et al. (1998) Carcinogenesis 19, 253-257; Davidson et al. (2003) Biomarkers 8, 51-61, all of which are hereby incorporated by reference. Although RNA is generally less suitable than DNA because it is readily degraded, it has previously been demonstrated that intact fecal eukaryotic mRNA can be isolated because of the presence of viable exfoliated colonocytes in the fecal stream as described in Albaugh (1992) International Journal of Cancer 52, 347-350; Davidson et al. (1995) Cancer Epidemiology Biomarkers and Prevention 4, 643-647; Davidson et al. (2003) Biomarkers 8, 51-61; Santiago et al. (2003) Journal of Virology 77, 2233-2242 and Kanaoka et al. (2004) Gastroenterology 127, 422-427, all of which are incorporated herein by reference.

Using exfoliated colonocytes, the discriminative mRNA expression signatures between conditions associated with inflammatory bowel disease versus normal conditions as well as conditions consistent with the presence of adenoma versus normal conditions has been described in Davidson et al. (2003) Biomarkers 8, 51-61. Those data suggest that mRNA isolated from exfoliated human colonocytes can be used to detect early stages of colon cancer, and possibly chronic inflammation. However, the microarray gene expression profile-based classification of colonic diseases for diagnostic purposes has yet to be solved. Therefore, a further embodiment of the present invention is the utilization of non-invasive mRNA procedures in patients at high risk for colorectal adenoma recurrence. In some embodiments, the effect of a legume enriched, low glycemic index (GI), high fermentable fiber diet, on subjects exhibiting a combination of risk factors including insulin resistance and history of adenomatous polyps is evaluated. This method evaluates the effects of legumes or a low GI diet on changes in intestinal gene expression profiles using exfoliated colonocytes. A further embodiment of the present invention involves the implementation of diagnostic gene sets (combinations) analyses for the objective classification of different phenotypes. These methods allow for the identification of both individual genes and two- to three-gene combinations for distinguishing polyps, insulin resistance, and exposure to a legume diet. The disclosed methods further reduce the classification error rate, with two and three-gene combinations providing robust classifiers that non-invasively identify discriminative signatures for diagnostic purposes.

### EXAMPLES

The following examples are provided in order to demonstrate and further illustrate certain preferred embodiments and aspects of the present invention and are not to be construed as limiting the scope thereof.

In the experimental disclosure that follows, the following abbreviations apply: bl1 (base line 1); bl2 (base line 2); dp1 (diet period 1); dp2 (diet period 2); GI (glycemic index); IR (insulin resistance); mRNA (messenger RNA); RMR (resting metabolic rate).

### Example I

### Materials and Methods

The effects of a legume enriched, low glycemic index, high fermentable fiber diet, were evaluated in participants with four possible combinations of risk factors, including insulin resistance (IR) and a history of adenomatous polyps. In a randomized, crossover, design-controlled feeding study, each participant consumed the “experimental diet”, defined as 1.5 cups of cooked dry beans per day, as well as a “control diet”, defined as an isocaloric average American diet, for four weeks, with a three-week washout period between diets. A total of 68 male subjects were examined, with 17 males assigned to each of four groups: Group 1 (previous history of adenomas and IR); Group 2 (previous history of adenomas without IR); Group 3 (IR with no history of adenomas); and Group 4 (non-IR and no history of adenomas). The effects of patient risk and diet on global gene expression profiling were examined using exfoliated colonic cells collected from the male subjects. All procedures used in the study were reviewed and approved by the human subjects' committees at the Pennsylvania State University and the National Institutes of Health. Study procedures are briefly summarized below.

### Subject Recruitment

Subjects were recruited with the assistance of gastroenterologists performing colonoscopies at the Mount Nittany Medical Center in State College, Pa. After receiving informed consent, the subject's height, weight and blood pressure were checked by study staff or the nurses at the clinic and a fasting blood sample was taken to determine overall health including fasting insulin and glucose to determine insulin sensitivity and cholesterol levels and lab tests for heart and liver function. A physician reviewed the results to determine eligibility for participation, with eligible consented participants asked to return to assess their resting metabolic rate (RMR). Each participant completed demographic, health and lifestyle questionnaires and subsequently provided instructions for completing a four-day food record for the purpose of estimating pre-study, baseline dietary intake.

### Inclusion and Exclusion Criteria

Eligible participants for the study were males between 35-75 years of age, with a body mass index of 25.0-34.9 kg/m², and having previously undergone a screening colonoscopy within the past two years. Subjects were selected that lacked pre-existing medical conditions including but not limited to cancer, heart disease, kidney disease and diabetes as well as a family history of such conditions, including but not limited to colorectal cancer, surgical resection of adenomas, bowel resection, polyposis syndrome and inflammatory bowel disease. Subjects were not permitted to take any medication that would alter inflammation markers, insulin, glucose, or blood lipids.

### Dietary Intervention

Subjects consumed one meal per day (breakfast or dinner) on site during the weekdays and consumed a packed lunch, snack and an additional meal at a time and place of convenience. Weekend meals were prepared and packed for carry out. Compliance was monitored according to procedures routinely used in the Pennsylvania State University General Clinical Center Research Center (GCRC). No foods other than those provided by the study kitchen were permitted. Alcohol consumption was limited to no more than two drinks/week during the controlled feeding period. A seven-day menu cycle was developed with a standard set of legumes of the Phaseolus vulgaris species, such as, navy beans, pinto beans, and kidney beans in order to limit nutrient and phytochemical differences in the seven-day diet cycle. The diet contained approximately 250 grams of legumes per day (1.5 cups). This level added approximately 20 grams of total dietary fiber and 8 g of soluble fiber/day. The diet was modified to provide other high glycemic index (GI) foods in the control diet so that the GI of the control diet had a GI of approximately 70 compared to a GI of 30 in the legume diet. Each daily menu was designed to maintain a constant level of fat (32-33 energy %), while the high legume low glycemic index diet had a total dietary fiber intake of approximately 40 grams per day compared to 20 grams per day for the high glycemic index diet. The protein level of both diets was approximately 18 energy %. In order to maintain the same level of red meat and fish (foods that have been associated with colon cancer) in both diets, the protein in legumes was substituted for protein from poultry. All nutrients were provided in amounts to meet the recommended dietary allowances for men of the same age groups. A food composite for each of the six days was freeze-dried and analyzed for macro-nutrient and fiber levels. Individual food items were purchased at the same time from the same supplier in order to assure uniformity of the diet.

mRNA Expression Microarray Analysis

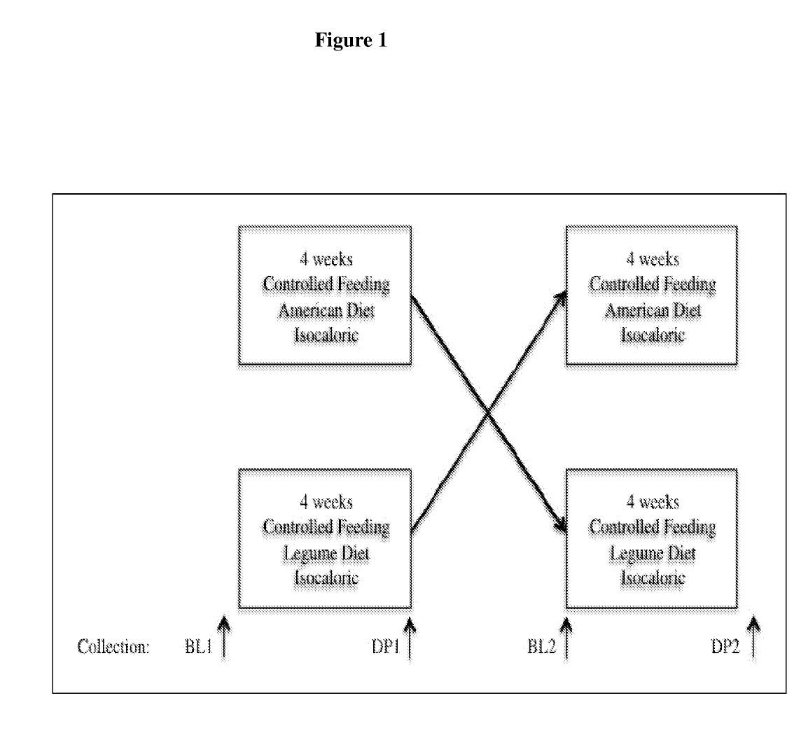

The overall study design is shown in FIG. 1. All fecal samples were processed within two hours of excretion, coded by the Research Assistant and stored at −80 degrees C. at the Penn State GCRC for later analysis. From each subject, poly A⁺RNA was isolated from feces as disclosed in Davidson et al. (1995) Cancer Epidemiology Biomarkers and Prevention 4, 643-647; Davidson et al. (1998) Carcinogenesis 19, 253-257; Davidson et al. (2003) Biomarkers 8, 51-61, all of which are hereby incorporated by reference. Due to the high level of bacterial RNA in fecal samples, poly A⁺RNA must be isolated in order to obtain a pure mammalian RNA population. As described in Davidson et al. (1995) Cancer Epidemiology Biomarkers and Prevention 4, 643-647, the isolation of poly A⁺ is free of bacterial RNA contamination. In addition, an Agilent 2100 Bioanalyzer was used to assess integrity of mucosal and fecal poly A⁺RNA. Samples were processed in strict accordance to the CodeLink™ Gene Expression Assay manual (Applied Microarray, Tempe, Ariz.) and analyzed using the Human whole Genome Expression Bioarray as provided for in Davidson et al. (2004) Cancer Research 64, 6797-6804, hereby incorporated by reference. Each array contains the entire human genome derived from publicly available, well-annotated mRNA sequences. This platform is unique because it is capable of detecting minimal differences in gene expression, as low as 1.3-fold with 95% confidence (Ramakrishnan, 2002; Stafford, 2003). The 3-D gel provides support for 30-mers in a matrix that holds the probe away from the surface of the slide. This substantially reduces background and enhances sensitivity, allowing for the detection of one transcript per cell with 50-200 ng of poly A⁺RNA (Stafford, 2003).

Arrays were inspected for spot morphology. Marginal spots were flagged as either background contamination (C) or irregular shape (I) in the output of the scanning software. Spots that passed the quality control standards were categorized as good (G). In addition, spots marked with (L) indicated a corresponding reading was “near the background”. The low (L) measurements reflect either true low gene expression levels or may have been caused by degradation of the mRNA resulting in a low signal. Samples collected from colonic mucosa previously exhibited a relatively low proportion (5-8%) of L spots as disclosed in Davidson et al. (2004) Cancer Research 64, 6797-6804, incorporated herein by reference. In contrast, the proportion of L spots in data obtained from fecal samples was significantly higher (65-83%).

### Microarray Data Normalization

The standard procedure for microarray data analysis requires a normalization step to facilitate the comparison of gene expression levels from two or more arrays. The goal of such a processing step is to reduce the technical variance while preserving the biologically meaningful variance produced by the different experimental conditions/treatments. The normalization procedures can be either “local” or “global” as disclosed in Quackenbush (2002) Nature Genetics Supplement 32, 496-501, incorporated in its entirety by reference. Besides these, model-based, parametric or non-parametric normalization procedures have been disclosed in Kerr et al. (2001) Genetic Research 77, 123-128; Sidorov et al. (2002) Information Sciences 146, 65-71; Bolstad et al. (2003) Bioinformatics 19, 185-193, all of which are incorporated herein by reference. However, none of these methods were developed for the situations where one deals with a high percentage of partially degraded mRNA in the samples. Recently, we proposed a two-stage normalization procedure for such data sets as described in Liu et al. (2005) Bioinformatics 21, 4000-4006, incorporated herein by reference. The method is built on non-parametric smoothing techniques with robustness consideration, and was used to evaluate the feasibility of properly extracting information from fecal mRNA data. We note, that the main objective of the two-stage normalization is to “regularize” the G spots for each gene while including the L spots that behave “similarly” to other G probes for that same gene, and excluding the outlying G probes. In contrast, our goal was to identify groups of genes/features that distinguish or classify between the different combinations of risk factors. Therefore, we adopted a conservative approach that does not include a normalization step, and focuses on a subset of genes that have been implicated in colorectal carcinogenesis. This procedure is justified by the observation that applying any kind of normalization to a data set with a high percentage of L spots has the potential to “flatten” the signal that results in a loss of data.

### Developing an Algorithm for Identifying Feature (Gene) Sets

Because there is high percentage of L spots on each array in the data set we first examined how the values of the parameters used by the CodeLink scanning software affect the number of G spots that are common for a subset of the arrays in our data set. To be specific, denoted by A^{k}ⱼ the set of genes xᵢ that have at most j raw mean spot intensity values less than where μ_{i,l}+kσ_{i,l} where μ_{i,l} is the value of local background median for the spot representing the gene xᵢ on the lth array, and σ_{i,l} is the corresponding standard deviation for that background signal. For example A^{1.5}₀ is the set of G spots that are common for all of the arrays in the data set (by default k=1.5 in the CodeLink software). Spots that are flagged C are not considered when the sets A^{k}ⱼ are formed. Notice that A^{k}ⱼ⊂A^{s}ᵣ if s≦k and j≦r. In particular, A^{k}ⱼ⊂A^{s}ⱼ, s≦k represents the fact that one gets a lesser number of common good spots if one requires a stronger signal as compared to the background. Also, A^{k}ⱼ⊂A^{k}ᵣ, j≦r represents the fact that the number of common genes increases if one allows more L spots per gene.

Keeping in mind that our main goal is to check if mRNA data from fecal colonocytes has the potential to classify different colon cancer risk factors we combined the so obtained sets A^{k}ⱼ with a set B of approximately 1300 known human colonic markers. Because our main goal was to determine if mRNA data from exfoliated colonocytes have the potential to classify different colon cancer risk factors, we compared the obtained array data sets (termed A) with a set of 529 putative human colonic markers (termed B; refer to Table V). Using such a prior biological knowledge we investigated the sets of common genes for A^{k}ⱼ and B. The number of those common genes for various values of the parameters k and j are given in Table VI. Based on these results, we focus on the intersection A²₁∩B. This conservative approach provides us with a subset of the known colonic biomarkers that have strong signal (k=2 compare to the CodeLink weaker default condition k=1.5) and no more than 1 low signal spot on the entire data set. One should notice that the microarray data could be grouped into various combinations of two different classes. This is due to the experimental design which lists to risk factors: (IR), and (−IR); four time points: Base line 1 (bl1), Diet period 1 (dp1), Base line 2 (bl2), Diet period 2 (dp2); and two diets: high legume low glycemic index, and control. These different groupings produce their respective sets A^{k}ⱼ that could be larger or smaller depending on which of the microarrays are included in the corresponding groups and classes (Table VII). Obviously, A^{k}ⱼ has the smallest possible size when one considers all of the data as being divided into two major categories, e.g. (+IR) vs (−IR). The next step in finding feature sets is to design classifiers that categorize samples based on the expression values of the genes from the intersection A²₁∩B. An important consideration is that the number of genes in such gene feature sets should be sufficiently small, and we construct the classifiers for feature sets of size 1, 2, and 3. There are two reasons why we desire classifiers involving small numbers of genes: (a) the limited number of samples often available in clinical studies makes classifier design and error estimation problematic for large feature sets as provided for in Dougherty et al. (2001) Comparative and Functional Genomics 2, 28-34, incorporated herein by reference, and (b) small gene sets facilitate design of practical immunohistochemical diagnostic panels. Thus, we use a simple linear discriminant analysis (LDA) classifier and a small number of genes. Given a set of features on which to base a classifier, one has to address not only the classifier design from sample data, but also the estimation of its error. When the number of potential feature sets is large, the key issue is whether a particular feature set provides good classification. A key concern is the precision with which the error of the designed classifier estimates the error of the optimal classifier. When data are limited, an error estimator may have a large variance and therefore may often be low. This can produce many feature sets and classifiers with low error estimates. The algorithm we use mitigates this problem by applying the bolstered error estimation as disclosed in Braga-Neto et al. (2004) Pattern Recognition 37, 1267-1281, incorporated in its entirety by reference. It has advantages with respect to commonly used error estimators such as re-substitution, cross-validation, and bootstrap methods for error estimation in terms of speed and accuracy (bias and variance). The basic idea is to bolster the original empirical distribution of the available data by means of suitable bolstering kernels placed at each datapoint location. The error can be computed analytically in some cases, such as in the case of LDA. The relatively small size of the set A²₁∩B allows for a comparing the errors of the potential feature sets of size 1, 2, and 3. The results of those comparisons are discussed in the next section.

### Results and Discussion

### Classification Analysis

In this feasibility study, our aim was to develop mRNA expression patterns that may establish the basis of a new non-invasive molecular diagnostic method. For this purpose, we applied an algorithm to 12 different pairs of classes arising from the experimental design as described in FIG. 1. The number of genes/features for each linear classifier was limited to three, which allowed for an exhaustive search. Biologists are often interested in finding individual genes that have some influence on the system under study. In the context of classification, this approach translates into finding single-gene classifiers. To illustrate how our approach compares to the traditional statistical analysis, we considered the classes (+IR, +Polyps) vs (−IR, −Polyps) at bl1. The top 10 feature sets of size 1 were compared to the differentially expressed genes in the set A²₁∩B, where t-tests were performed using the log₂-transformed raw intensity values. The comparison revealed that 7 out of the 10 top 1-feature sets (genes) identified by the linear (LDA) classifier also had p-values <0.05. This should not be surprising because individual, differentially expressed genes are often used to discriminate between phenotypes. The results disclosed herein show that there are several cases where single genes can provide good classification in terms of the error estimate. However, when comparing these results to the two-feature classification for the same two classes, a trend is observed as described in Martins et al. (2008) Journal of Selected Topics in Signal Processing 2, 424-439, incorporated in relevant parts by reference. The concept of intrinsically multivariate predictive (IMP) genes was introduced based on observations where expression profiles of a group of genes predicts the target, e.g. a gene or a phenotype) with great accuracy while any proper subset of these genes produces poor prediction.

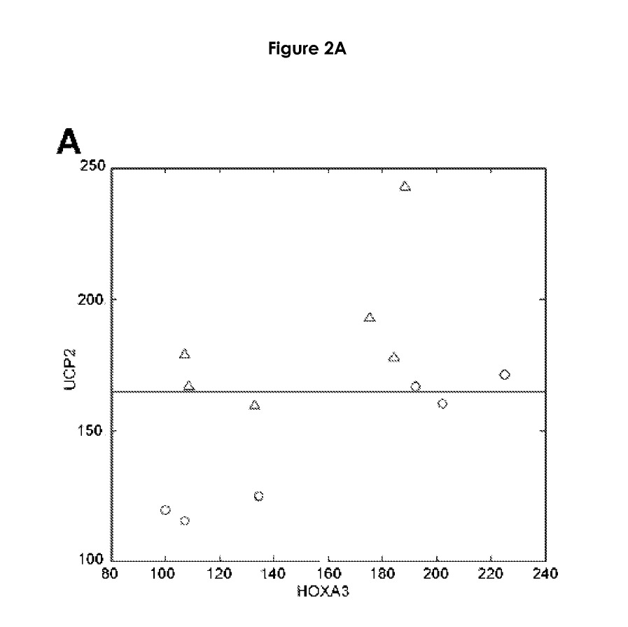

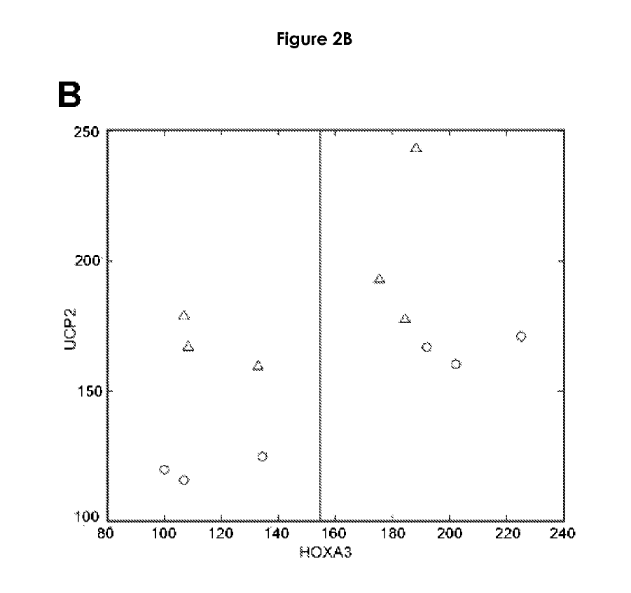

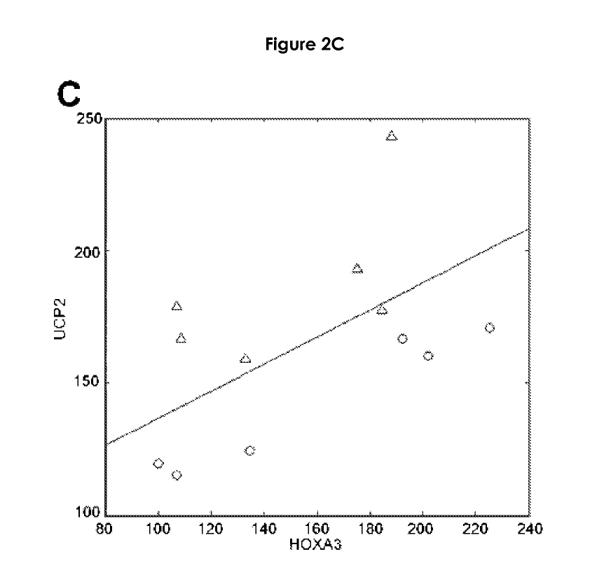

The concept of intrinsically multivariate predictive (IMP) genes is shown where expression profiles of a group of genes predict the phenotype. Results represent a linear classification of (+IR, +Polyps) subjects (◯) versus (−IR, −Polyps) subjects (Δ) at BL1. UCP2 and HOXA3 were used as individual one-feature sets (A and B) as compared with both genes together as a two-feature set (C). The bolstered error is 0.2784, 0.4882, and 0.1415 for (A), (B), and (C), respectively. Specifically, the expression profiles of a group of genes predicted the target (either a gene or a phenotype) with greater accuracy relative to any proper subset of these genes. For example, single-gene classifiers (one-feature) based on either the Homeoboxpr otein-A3 (HOXA3) or uncoupling protein-2 (UCP2) performed very poorly when discriminating between (+IR, +Polyps) and (−IR, −Polyps) at BL1 (Table II; FIGS. 2A and B). Interestingly, HOXA3 was close to the worst predictor of all of the available 97 genes (ranked 94). In comparison, when combined as a two-feature set, UCP2 and HOXA3 provided one of the best two-feature classifiers (one misclassified data point only) among all of the 4,656 possible two-gene sets (Table II; 3C). These data clearly illustrate why complex phenotypes can be explained better by multivariate feature sets.

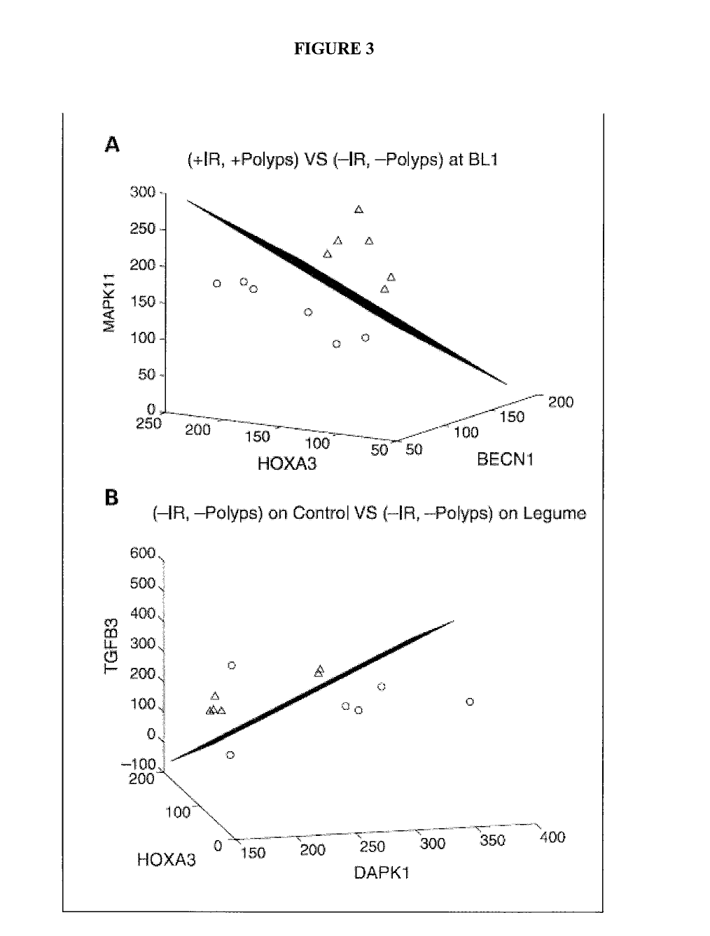

To identify sets of genes that perform in a multivariate manner to provide strong classification, we specifically looked for pairs of genes that performed better than either of the genes individually, and triplets of genes that performed well and substantially better than the best-performing pair among the three, and so on. To estimate the improvements of the classification performance, we introduced two quantities for each feature set: ε_{bolstered} and Δ(ε_{bolstered}). ε_{bolstered} denotes the bolstered resubstitution error for the LDA classifier for the respective feature set, and Δ(ε_{bolstered}) denotes the largest decrease in error for the full feature set relative to all of its subsets. The feature sets were initially ranked based on the value of ε_{bolstered}, and subsequently ranked again based on the improvement Δ(ε_{bolstered}). For multiple-gene classifiers, we focused on feature sets with high rank in both lists. Along these lines, we designed two-feature classifiers for the classification of (+IR, +Polyps) versus (−IR, −Polyps) data at baseline BL1; (−IR, −Polyps, control diet) versus (−IR, −Polyps, legume diet) data at the end of the two diet periods DP1 and DP2; (+IR, +Polyps) versus (−IR, −Polyps) at baselines BL1 and BL2; (+Polyps) versus (−Polyps) at baselines BL1 and BL2; and (+IR) versus (−IR) at all of the time points. Table II and Table III describe the best (according to this ranking procedure) feature sets identified for the first two of these classification categories, and FIGS. 3A and B shows representative multivariate classifiers.

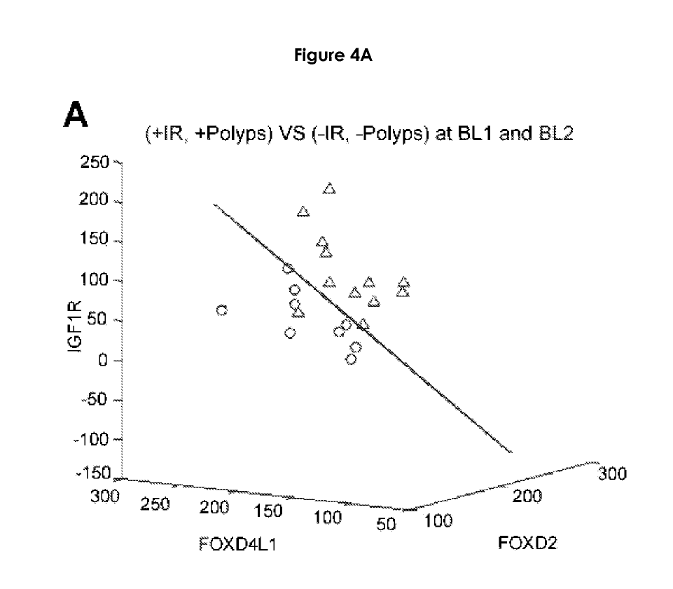

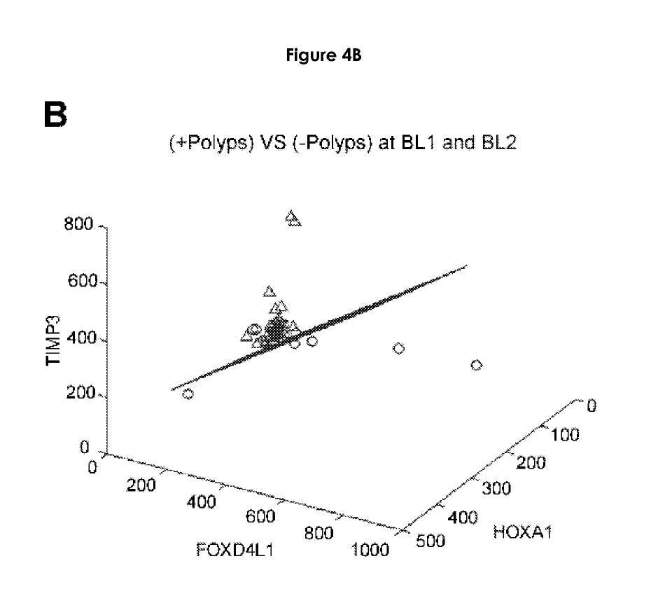

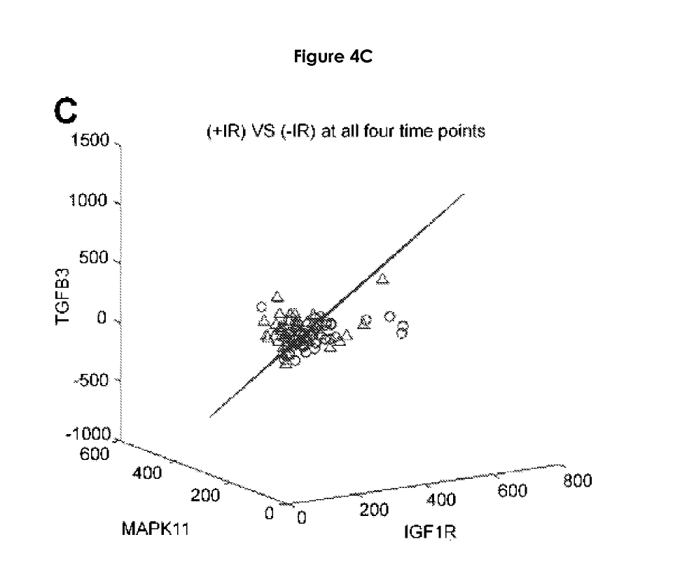

The results in FIG. 4 show that the two factors, IR and history of adenomas, should be considered in tandem when determining the risk for the patient. For example, combining baseline samples (BL1 and BL2) increased the classification error, indicating complications related to the crossover design (FIG. 4A). Similarly, the three-feature set LDA classifiers performed poorly when the classification was considered separately with respect to either one of the two experimental factors (IR) or (Polyps; FIGS. 4B and C). The advantage of reporting the results in this way is that multivariate discriminatory power is revealed. This is clearly shown in Table II with regard to HOXA3. The gene did not appear on the single-gene list, indicating that the error of the respective classifier exceeded 0.3 (εbolstered=0.4882). However, it appeared with UCP2, 14-3-3ζ (YWHAZ), insulin growth factor receptor-I (IGF1R), beclin-1 (BECN1), and mitogen-activated protein kinase-11 (MAPK11) genes in the two-gene and three-gene lists, which improved classification error. Interestingly, members of the homeoprotein family of transcription factors (HOXA3 and HOXC6) are developmental regulators of gastrointestinal growth, patterning, and differentiation (Fujiki K, Duerr E, Kikuchi H, et al. Hoxc6 is overexpressed in gastrointestinal carcinoids and interacts with JunD to regulate tumor growth. Gastroenterology 2008; 135:907-16). It is also noteworthy that YWHAZ and IGF1R are capable of regulating apoptosis and cell adhesion (Sekharam M, Zhao H, Sun M, et al. Insulin-like growth factor 1 receptor enhances invasion and induces resistance to apoptosis of colon cancer cells through the Akt/Bcl-xL pathway. Cancer Res 2003; 63:7708-16, Niemantsverdriet M, Wagner K, Visser M, Backendorf C. Cellular functions of 14-3-3ζ in apoptosis and cell adhesion emphasize its oncogenic character. Oncogene 2008; 27:1315-9); UCP2 promotes chemoresistance in cancer cells and mitochondrial Ca2+ sequestration; BECN1 stimulates autophagy and inhibits tumor cell growth (Pattingre S, Espert L, Biard-Piechaczyk M, Codogno P. Regulation of macroautophagy by mTOR and Beclin 1 complexes. Biochimie 2008; 90:313-23); and MAPK11 (p38β) mediates response to inflammatory cytokines and cellular stress (Beardmore V A, Hinton H J, Eftychi C, et al. Generation and characterization of p38β (MAPK11) gene-targeted mice. Mol Cell Biol 2005; 25:10454-64). For comparative purposes, fold changes in select genes are presented in Table VIII.

TABLE I

| | Sample | Common Genes |
|---|---|---|
| Classification Groups | Size | in A₁² ∩C |
| (+IR, +Polyps) VS (−IR, −Polyps) at BL1 | 12 | 97 |
| (+IR, +Polyps) on Control VS (+IR, +Polyps) on Legume | 11 | 103 |
| (−IR, −Polyps) on Control VS (−IR, −Polyps) on Legume | 12 | 145 |
| (+IR, +Polyps) on Control VS (−IR, −Polyps) on Control | 11 | 121 |
| (+IR, +Polyps) on Legume VS (−IR, −Polyps) on Legume | 12 | 114 |
| (+IR, +Polyps) VS (−IR, −Polyps) at BL1 & BL2 | 21 | 92 |
| (+Polyps) VS (−Polyps) at BL1 | 23 | 64 |
| (+IR) VS (−IR) at BL1 | 23 | 64 |
| (+Polyps) VS (−Polyps) at BL1 & BL2 | 41 | 59 |
| (+Polyps) on Control VS (+Polyps) on Legume | 21 | 87 |
| (+IR) on Control VS (+IR) on Legume | 23 | 74 |
| (+IR) VS (−IR) at all time points | 86 | 54 |

TABLE II

| Gene names | ε_{bolstered} | Δ(ε_{bolstered}) |
|---|---|---|
| IGF1R | 0.1094 | |
| CDK4 | 0.1200 | |
| BECN1 | 0.1223 | |
| NOS3 | 0.1436 | |
| ALOX12B | 0.1477 | |
| NOS3, WNT1 | 0.1277 | 0.2656 |
| HOXA3, UCP2 | 0.1415 | 0.3467 |
| IGF1R, WNT1 | 0.1484 | 0.2449 |
| ID2, IGF1R | 0.1486 | 0.3139 |
| HOXA3, YWHAZ | 0.1503 | 0.3379 |
| HOXA3, IGF1R | 0.1513 | 0.3369 |
| BECN1, HOXA3, MAPK11 | 0.0891 | 0.3991 |
| BECN1, HOXA3, IGF1R | 0.0907 | 0.3975 |
| HOXA3, MAPK11, YWHAZ | 0.0935 | 0.3947 |
| HOXA3, HOXC6, MAPK11 | 0.0941 | 0.3941 |
| HOXA3, MAPK11, NOS3 | 0.0987 | 0.3895 |
| HOXA3, UCP2, YWHAZ | 0.1001 | 0.3881 |
| HOXA3, IGF1R, YWHAZ | 0.1006 | 0.3876 |
| BECN1, DAPK1, IGF1R | 0.1012 | 0.3768 |
| HOXA3, HOXC6, TJP1 | 0.1023 | 0.3859 |
| HOXA3, HOXC6, IGF1R | 0.1079 | 0.3803 |

TABLE III

| Gene names | ε_{bolstered} | Δ(ε_{bolstered}) |
|---|---|---|
| TGFB3 | 0.2350 | |
| FOXP4 | 0.2586 | |
| TP53 | 0.2970 | |
| BAD | 0.3009 | |
| FOXO1A | 0.3033 | |
| DAPK1, HOXA3 | 0.1829 | 0.3760 |
| BAD, LYZL6 | 0.2275 | 0.2321 |
| IGF1R, LEF1 | 0.2315 | 0.2488 |
| DAPK1, FOXM1 | 0.2371 | 0.2336 |
| IGF2, TGFB3 | 0.2455 | 0.2814 |
| LEF1, TGFB3 | 0.2459 | 0.2344 |
| DAPK1, TP53 | 0.2642 | 0.2426 |
| APC, CDC42 | 0.2650 | 0.2564 |
| DAPK1, HOXA3, TGFB3 | 0.1675 | 0.3914 |
| DAPK1, LEF1, TGFB3 | 0.1799 | 0.3004 |
| DAPK1, HOXA3, LEF1 | 0.1854 | 0.3735 |
| DAPK1, HOXA3, SELP | 0.1887 | 0.3702 |
| CAMK2A, DAPK1, HOXA3 | 0.1922 | 0.3667 |
| DAPK1, HOXA3, SPARC | 0.1944 | 0.3645 |
| DAPK1, HOXA3, PRKACG | 0.1969 | 0.3620 |
| DAPK1, HOXA3, SFRP5 | 0.1982 | 0.3607 |
| BAD, FOXE3, PTK2 | 0.2003 | 0.3018 |
| CA5B, DAPK1, HOXA3 | 0.2028 | 0.3561 |
| CD44, DAPK1, HOXA3 | 0.2052 | 0.3537 |
| BAD, FOXP4, GSS | 0.2056 | 0.3112 |
| BAD, FOXE3, PTK2B | 0.2072 | 0.3187 |
| APC2, DAPK1, HOXA3 | 0.2117 | 0.3472 |

TABLE IV

| Subject ID | Study Group | BL 1 | End DP 1 | BL 2 | End DP 2 | Subject ID | DP1 | DP2 |
|---|---|---|---|---|---|---|---|---|
| LEG 01 | 3 | x | x | x | x | LEG 01 | L | C |
| LEG 02 | 2 | x | x | x | x | LEG 02 | C | L |
| LEG 03 | 1 | x | x | x | x | LEG 03 | C | L |
| LEG 04 | 3 | x | x | x | x | LEG 04 | L | C |
| LEG 05 | 2 | x | x | x | x | LEG 05 | L | C |
| LEG 06 | 4 | x | x | x | x | LEG 06 | C | L |
| LEG 08 | 3 | x | x | x | x | LEG 08 | C | L |
| LEG 09 | 2 | x | x | x | x | LEG 09 | L | C |
| LEG 10 | 2 | x | x | m | x | LEG 10 | C | L |
| LEG 11 | 4 | x | x | x | x | LEG 11 | L | C |
| LEG 13 | 1 | x | x | m | x | LEG 13 | C | L |
| LEG 14 | 3 | x | x | x | x | LEG 14 | C | L |
| LEG 18 | 3 | x | x | x | x | LEG 18 | L | C |
| LEG 19 | 4 | x | x | x | x | LEG 19 | C | L |
| LEG 24 | 4 | x | x | x | x | LEG 24 | L | C |
| LEG 26 | 4 | x | x | x | x | LEG 26 | C | L |
| LEG 27 | 4 | x | x | x | x | LEG 27 | L | C |
| LEG 33 | 2 | x | x | m | x | LEG 33 | C | L |
| LEG 44 | 1 | x | x | m | x | LEG 44 | L | C |
| LEG 47 | 1 | x | x | x | x | LEG 47 | C | L |
| LEG 49 | 1 | x | x | m | x | LEG 49 | L | C |
| LEG 54 | 1 | x | x | x | m | LEG 54 | L | C |
| LEG 65 | 3 | x | x | x | x | LEG 65 | C | L |

x = array was processed;
m = missing sample
L = Legume diet:
C = American control diet
Study Group:
1 = +insulin resistance/+polyps;
2 = −insulin resistance/+polyps
3 = +insulin resistance/−polyps:
4 = −insulin resistance/−polyps

TABLE V

| | | Gene |
|---|---|---|
| PROBE_NAME | NCBI_ACC | abbreviation |
| GE55294 | NM_012138.2 | AATF |
| GE57835 | NM_000927.3 | ABCB1 |
| GE82449 | NM_018849.1 | ABCB4 |
| GE60435 | NM_000017.1 | ACADS |
| GE54239 | NM_004458.1 | ACSL4 |
| GE59148 | NM_003816.2 | ADAM9 |
| GE53921 | NM_006988.3 | ADAMTS1 |
| GE61341 | NM_005099.3 | ADAMTS4 |
| GE80938 | NM_000697.1 | ALOX12 |
| GE54325 | NM_001139.1 | ALOX12B |
| GE80985 | NM_001140.3 | ALOX15 |
| GE59425 | NM_001141.1 | ALOX15B |
| GE57484 | NM_000698.1 | ALOX5 |
| GE54404 | NM_005503.2 | APBA2 |
| GE58061 | NM_000038.3 | APC |
| GE53318 | NM_005883.1 | APC2 |
| GE58776 | NM_001641.2 | APEX1 |
| GE59705 | NM_000045.2 | ARG1 |
| GE59481 | NM_001172.3 | ARG2 |
| GE62666 | NM_005172.1 | ATOH1 |
| GE57035 | NM_001686.3 | ATP5B |
| GE53329 | NM_006095.1 | ATP8A1 |
| GE61790 | NM_003502.2 | AXIN1 |
| GE58341 | NM_004655.1 | AXIN2 |
| GE61740 | NM_032989.1 | BAD |
| GE60444 | NM_001188.2 | BAK1 |
| GE57679 | NM_004324.3 | BAX |
| GE79818 | NM_003921.2 | BCL10 |
| GE62993 | NM_004049.2 | BCL2A1 |
| GE60374 | NM_138578.1 | BCL2L1 |
| GE80623 | NM_020396.2 | BCL2L10 |
| GE890567 | NM_138622.2 | BCL2L11 |
| GE83665 | NM_138639.1 | BCL2L12 |
| GE61161 | NM_015367.2 | BCL2L13 |
| GE83170 | NM_030766.1 | BCL2L14 |
| GE57412 | NM_004050.2 | BCL2L2 |
| GE79828 | NM_003766.2 | BECN1 |
| GE59117 | NM_001166.3 | BIRC2 |
| GE612545 | AI873224.1 | BIRC4 |
| GE59394 | NM_001168.1 | BIRC5 |
| GE57870 | NM_001200.1 | BMP2 |
| GE59170 | NM_001202.2 | BMP4 |
| GE55306 | NM_017589.2 | BTG4 |
| GE58358 | NM_005181.2 | CA3 |
| GE58106 | NM_000717.2 | CA4 |
| GE58264 | NM_001739.1 | CA5A |
| GE53590 | NM_007220.2 | CA5B |
| GE53630 | NM_015981.2 | CAMK2A |
| GE62184 | NM_001221.2 | CAMK2D |
| GE58946 | NM_032991.1 | CASP3 |
| GE61602 | NM_001752.1 | CAT |
| GE54670 | NM_001753.3 | CAV1 |
| GE57886 | NM_031966.2 | CCNB1 |
| GE795552 | AW439398.1 | CCND1 |
| GE79118 | NM_013230.1 | CD24 |
| GE59805 | NM_001001389.1 | CD44 |
| GE62023 | NM_212530.1 | CDC25B |
| GE57603 | NM_001791.2 | CDC42 |
| GE57732 | NM_004360.2 | CDH1 |
| GE59262 | NM_001257.2 | CDH13 |
| GE54070 | NM_004642.2 | CDK2AP1 |
| GE57828 | NM_000075.2 | CDK4 |
| GE58861 | NM_000389.2 | CDKN1A |
| GE81344 | NM_004064.2 | CDKN1B |
| GE80886 | NM_000076.1 | CDKN1C |
| GE59038 | NM_058195.2 | CDKN2A |
| GE81459 | L36844.1 | CDKN2B |
| GE79397 | NM_001804.1 | CDX1 |
| GE59216 | NM_001265.2 | CDX2 |
| GE59735 | NM_001712.2 | CEACAM1 |
| GE62998 | NM_001815.1 | CEACAM3 |
| GE81072 | NM_001817.1 | CEACAM4 |
| GE81388 | NM_004363.1 | CEACAM5 |
| GE79541 | NM_002483.3 | CEACAM6 |
| GE54005 | NM_006890.1 | CEACAM7 |
| GE579012 | NM_001816.2 | CEACAM8 |
| GE87279 | NM_020219.2 | CEAL1 |
| GE742535 | NM_033377.1 | CGB1 |
| GE62911 | NM_033142.1 | CGB7 |
| GE54896 | AY358652.1 | CLDN1 |
| GE79293 | NM_001306.2 | CLDN3 |
| GE54025 | NM_003277.2 | CLDN5 |
| GE56220 | NM_199328.1 | CLDN8 |
| GE54150 | NM_130444.1 | COL18A1 |
| GE80889 | NM_000089.3 | COL1A2 |
| GE59553 | NM_001861.2 | COX4I1 |
| GE81079 | NM_001862.2 | COX5B |
| GE61834 | NM_015513.2 | CRELD1 |
| GE61173 | NM_004385.2 | CSPG2 |
| GE54206 | NM_004386.1 | CSPG3 |
| GE60186 | NM_001897.3 | CSPG4 |
| GE54549 | NM_006574.2 | CSPG5 |
| GE54158 | NM_005445.2 | CSPG6 |
| GE60116 | NM_001904.2 | CTNNB1 |
| GE63376 | NM_001511.1 | CXCL1 |
| GE60012 | NM_004938.1 | DAPK1 |
| GE54486 | NM_014326.2 | DAPK2 |
| GE53147 | NM_001348.1 | DAPK3 |
| GE79017 | NM_020548.4 | DBI |
| GE60014 | NM_005215.1 | DCC |
| GE55123 | NM_004942.2 | DEFB4 |
| GE53874 | NM_015881.4 | DKK3 |
| GE61043 | NM_004413.1 | DPEP1 |
| GE54709 | NM_004147.3 | DRG1 |
| GE60381 | NM_001943.1 | DSG2 |
| GE56426 | NM_198057.1 | DSIPI |
| GE61271 | NM_004417.2 | DUSP1 |
| GE59081 | NM_001948.2 | DUT |
| GE59467 | U80811.1 | EDG2 |
| GE54109 | NM_004720.4 | EDG4 |
| GE61188 | NM_012152.1 | EDG7 |
| GE56653 | NM_004429.3 | EFNB1 |
| GE58272 | NM_001963.2 | EGF |
| GE59632 | NM_005228.3 | EGFR |
| GE59464 | NM_015409.2 | EP400 |
| GE80071 | NM_004441.3 | EPHB1 |
| GE54191 | NM_004442.5 | EPHB2 |
| GE62563 | NM_004444.4 | EPHB4 |
| GE57332 | NM_004445.2 | EPHB6 |
| GE54479 | NM_194356.1 | EPIM |
| GE57810 | NM_004448.2 | ERBB2 |
| GE59651 | NM_000125.1 | ESR1 |
| GE62899 | NM_016946.3 | F11R |
| GE81021 | NM_001443.1 | FABP1 |
| GE79151 | NM_000134.2 | FABP2 |
| GE80868 | NM_004102.2 | FABP3 |
| GE79086 | NM_001444.1 | FABP5 |
| GE55154 | NM_001446.3 | FABP7 |
| GE60092 | NM_003824.2 | FADD |
| GE59061 | NM_004104.4 | FASN |
| GE62924 | NM_004111.4 | FEN1 |
| GE54695 | NM_003862.1 | FGF18 |
| GE79526 | NM_005130.3 | FGFBP1 |
| GE59130 | NM_004496.2 | FOXA1 |
| GE80477 | NM_021784.3 | FOXA2 |
| GE81402 | NM_004497.2 | FOXA3 |
| GE81766 | NM_012182.1 | FOXB1 |
| GE80442 | NM_001453.1 | FOXC1 |
| GE61182 | D63042.1 | FOXC2 |
| GE59268 | NM_004472.1 | FOXD1 |
| GE80316 | NM_004474.2 | FOXD2 |
| GE614874 | NM_012183.1 | FOXD3 |
| GE561268 | NM_207305.1 | FOXD4 |
| GE59533 | NM_004473.3 | FOXE1 |
| GE81767 | NM_012186.1 | FOXE3 |
| GE61775 | NM_001451.1 | FOXF1 |
| GE58940 | NM_001452.1 | FOXF2 |
| GE59988 | NM_005249.3 | FOXG1B |
| GE79568 | NM_003923.1 | FOXH1 |
| GE57620 | NM_012188.3 | FOXI1 |
| GE817567 | AI684913.1 | FOXJ1 |
| GE56857 | NM_018416.2 | FOXJ2 |
| GE53710 | NM_014947.3 | FOXJ3 |
| GE61025 | NM_181431.1 | FOXK2 |
| GE481743 | NM_005250.1 | FOXL1 |
| GE79861 | NM_023067.2 | FOXL2 |
| GE59391 | NM_021953.2 | FOXM1 |
| GE80086 | NM_003593.2 | FOXN1 |
| GE573609 | NM_213596.1 | FOXN4 |
| GE54250 | NM_002015.2 | FOXO1A |
| GE54251 | NM_001455.2 | FOXO3A |
| GE620465 | AI732568.1 | FOXP1 |
| GE771702 | NM_148899.1 | FOXP2 |
| GE503510 | NM_014009.2 | FOXP3 |
| GE55167 | BM679319.1 | FOXP4 |
| GE83634 | NM_033260.2 | FOXQ1 |
| GE573639 | NM_181721.1 | FOXR1 |
| GE525901 | NM_198451.1 | FOXR2 |
| GE61934 | NM_002029.3 | FPR1 |
| GE81028 | NM_001469.3 | G22P1 |
| GE59654 | NM_000402.2 | G6PD |
| GE57684 | NM_001924.2 | GADD45A |
| GE82030 | NM_015675.1 | GADD45B |
| GE62138 | NM_006705.2 | GADD45G |
| GE81449 | NM_004864.1 | GDF15 |
| GE80927 | NM_000581.2 | GPX1 |
| GE81111 | NM_002083.2 | GPX2 |
| GE695561 | AW129281.1 | GPX3 |
| GE81112 | NM_002084.2 | GPX3 |
| GE81113 | NM_002085.1 | GPX4 |
| GE80647 | NM_003996.2 | GPX5 |
| GE54767 | NM_015696.2 | GPX7 |
| GE79962 | AF154054.1 | GREM1 |
| GE88366 | CA437861.1 | GREM2 |
| GE61362 | NM_000177.3 | GSN |
| GE54991 | NM_000637.2 | GSR |
| GE59100 | NM_000178.2 | GSS |
| GE79099 | NM_145740.2 | GSTA1 |
| GE503167 | AI762244.1 | GSTA2 |
| GE54166 | NM_000847.3 | GSTA3 |
| GE61329 | NM_001512.2 | GSTA4 |
| GE516052 | NM_153699.1 | GSTA5 |
| GE61688 | NM_000561.2 | GSTM1 |
| GE835116 | NM_000849.3 | GSTM3 |
| GE57545 | NM_000851.2 | GSTM5 |
| GE61334 | NM_000852.2 | GSTP1 |
| GE60049 | NM_000853.1 | GSTT1 |
| GE79395 | NM_033553.2 | GUCA2A |
| GE79398 | NM_007102.1 | GUCA2B |
| GE537019 | NM_005524.2 | HES1 |
| GE62425 | NM_000601.3 | HGF |
| GE82322 | NM_018194.1 | HHAT |
| GE59057 | NM_001530.2 | HIF1A |
| GE60472 | NM_005338.4 | HIP1 |
| GE568338 | NM_003493.2 | HIST3H3 |
| GE659143 | NM_145904.1 | HMGA1 |
| GE57571 | NM_000191.1 | HMGCL |
| GE59927 | NM_002130.4 | HMGCS1 |
| GE60087 | NM_005518.2 | HMGCS2 |
| GE61857 | NM_002133.1 | HMOX1 |
| GE79731 | NM_005522.3 | HOXA1 |
| GE88138 | NM_153715.1 | HOXA10 |
| GE79995 | NM_005523.4 | HOXA11 |
| GE80920 | NM_000522.2 | HOXA13 |
| GE667398 | NM_006735.3 | HOXA2 |
| GE83160 | NM_030661.3 | HOXA3 |
| GE80064 | NM_002141.2 | HOXA4 |
| GE57892 | NM_019102.2 | HOXA5 |
| GE479776 | NM_024014.2 | HOXA6 |
| GE483668 | NM_006896.2 | HOXA7 |
| GE54105 | NM_002142.3 | HOXA9 |
| GE59739 | NM_002144.2 | HOXB1 |
| GE81628 | NM_006361.2 | HOXB13 |
| GE59738 | NM_002145.2 | HOXB2 |
| GE59740 | NM_002146.3 | HOXB3 |
| GE56717 | NM_024015.3 | HOXB4 |
| GE58143 | NM_002147.2 | HOXB5 |
| GE82457 | NM_156036.1 | HOXB6 |
| GE81403 | NM_004502.2 | HOXB7 |
| GE79102 | NM_024016.2 | HOXB8 |
| GE58746 | NM_017409.2 | HOXC10 |
| GE497436 | NM_014212.2 | HOXC11 |
| GE808308 | NM_173860.1 | HOXC12 |
| GE80244 | NM_017410.2 | HOXC13 |
| GE59694 | NM_014620.2 | HOXC4 |
| GE59871 | NM_018953.2 | HOXC5 |
| GE57846 | NM_153693.1 | HOXC6 |
| GE812007 | NM_022658.3 | HOXC8 |
| GE55497 | NM_006897.1 | HOXC9 |
| GE82840 | NM_024501.1 | HOXD1 |
| GE81125 | NM_002148.2 | HOXD10 |
| GE82602 | NM_021192.2 | HOXD11 |
| GE729613 | NM_021193.2 | HOXD12 |
| GE80921 | NM_000523.2 | HOXD13 |
| GE881059 | BQ941558.1 | HOXD4 |
| GE59727 | NM_019558.2 | HOXD8 |
| GE61129 | NM_014213.2 | HOXD9 |
| GE57789 | NM_000860.3 | HPGD |
| GE57461 | NM_005343.2 | HRAS |
| GE53171 | BC041412.2 | HSPA12A |
| GE811123 | BX116887.1 | HSPA12B |
| GE82101 | NM_016299.1 | HSPA14 |
| GE81493 | AK097113.1 | HSPA1A |
| GE81494 | NM_005346.3 | HSPA1B |
| GE62810 | NM_000201.1 | ICAM1 |
| GE81127 | NM_002166.4 | ID2 |
| GE59822 | NM_000618.2 | IGF1 |
| GE80962 | NM_000875.2 | IGF1R |
| GE79183 | NM_000612.2 | IGF2 |
| GE508441 | BC034757.1 | IHH |
| GE54386 | W52507.1 | IKBKAP |
| GE62527 | NM_001556.1 | IKBKB |
| GE61317 | NM_014002.2 | IKBKE |
| GE54691 | NM_003639.2 | IKBKG |
| GE61058 | NM_000572.2 | IL10 |
| GE80144 | NM_000641.2 | IL11 |
| GE563350 | NM_000575.3 | IL1A |
| GE79235 | NM_000576.2 | IL1B |
| GE902998 | NM_000586.2 | IL2 |
| GE63369 | NM_020525.4 | IL22 |
| GE53760 | NM_016584.2 | IL23A |
| GE59635 | NM_000417.1 | IL2RA |
| GE59660 | NM_000600.1 | IL6 |
| GE61911 | NM_002211.2 | ITGB1 |
| GE54170 | NM_181468.1 | ITGB4BP |
| GE59788 | NM_002231.2 | KAI1 |
| GE61879 | NM_000238.2 | KCNH2 |
| GE58431 | NM_007035.2 | KERA |
| GE53820 | BG912905.1 | KIAA1199 |
| GE59676 | NM_000222.1 | KIT |
| GE86000 | NM_004235.3 | KLF4 |
| GE61031 | NM_033360.2 | KRAS2 |
| GE79418 | BU153499.1 | KRT8 |
| GE60355 | NM_005562.1 | LAMC2 |
| GE80131 | NM_002295.2 | LAMR1 |
| GE80813 | NM_002300.3 | LDHB |
| GE56173 | NM_016269.2 | LEF1 |
| GE60548 | NM_002306.1 | LGALS3 |
| GE57621 | NM_005567.2 | LGALS3BP |
| GE61853 | NM_003667.2 | LGR5 |
| GE60093 | NM_013975.1 | LIG3 |
| GE61118 | NM_000627.2 | LTBP1 |
| GE54721 | NM_006330.2 | LYPLA1 |
| GE79416 | NM_000239.1 | LYZ |
| GE87934 | NM_032517.3 | LYZL1 |
| GE573861 | NM_144634.2 | LYZL4 |
| GE54752 | NM_020426.1 | LYZL6 |
| GE534117 | NM_002745.2 | MAPK1 |
| GE62730 | NM_002753.2 | MAPK10 |
| GE88364 | NM_002751.5 | MAPK11 |
| GE62595 | NM_002969.3 | MAPK12 |
| GE54058 | NM_002754.3 | MAPK13 |
| GE57735 | NM_001315.1 | MAPK14 |
| GE79221 | NM_002746.1 | MAPK3 |
| GE84984 | R87970.1 | MAPK4 |
| GE79439 | NM_002748.2 | MAPK6 |
| GE81188 | NM_139033.1 | MAPK7 |
| GE57700 | NM_139046.1 | MAPK8 |
| GE81189 | NM_002752.3 | MAPK9 |
| GE59858 | NM_002382.3 | MAX |
| GE57107 | NM_004526.2 | MCM2 |
| GE80542 | NM_130799.1 | MEN1 |
| GE57468 | J02958.1 | MET |
| GE57913 | NM_002412.2 | MGMT |
| GE80049 | NM_145792.1 | MGST1 |
| GE63001 | NM_032390.3 | MKI67IP |
| GE58891 | NM_000249.2 | MLH1 |
| GE80495 | NM_002421.2 | MMP1 |
| GE518683 | NM_002427.2 | MMP13 |
| GE79854 | NM_002422.2 | MMP3 |
| GE57525 | NM_004994.1 | MMP9 |
| GE81153 | NM_002434.1 | MPG |
| GE58211 | NM_000251.1 | MSH2 |
| GE59286 | NM_002439.1 | MSH3 |
| GE60542 | NM_000179.1 | MSH6 |
| GE53323 | NM_002442.2 | MSI1 |
| GE88536 | N26272.1 | MSI2 |
| GE57533 | NM_182741.1 | MUC1 |
| GE81158 | NM_002457.1 | MUC2 |
| GE86707 | AB038784.1 | MUC3B |
| GE82366 | NM_018406.2 | MUC4 |
| GE60413 | AJ001402.1 | MUC5AC |
| GE564339 | U78550.1 | MUC5B |
| GE59609 | AK096772.1 | MUC6 |
| GE57582 | NM_005962.3 | MXI1 |
| GE57537 | NM_002467.2 | MYC |
| GE81665 | NM_006656.4 | NEU3 |
| GE57992 | NM_003998.2 | NFKB1 |
| GE81163 | NM_002502.2 | NFKB2 |
| GE583888 | NM_145285.1 | NKX2-3 |
| GE58970 | NM_000620.1 | NOS1 |
| GE61880 | NM_000625.3 | NOS2A |
| GE57703 | NM_000603.3 | NOS3 |
| GE58433 | NM_007052.3 | NOX1 |
| GE57330 | NM_002519.1 | NPAT |
| GE79523 | NM_002524.2 | NRAS |
| GE59456 | NM_002528.4 | NTHL1 |
| GE62798 | NM_004822.1 | NTN1 |
| GE79384 | NM_006183.3 | NTS |
| GE79065 | NM_002538.2 | OCLN |
| GE59733 | NM_002539.1 | ODC1 |
| GE58739 | NM_016819.2 | OGG1 |
| GE80094 | NM_002583.2 | PAWR |
| GE61312 | NM_002592.2 | PCNA |
| GE61229 | NM_002634.2 | PHB |
| GE55121 | NM_004570.2 | PIK3C2G |
| GE85962 | NM_181523.1 | PIK3R1 |
| GE57869 | NM_000300.2 | PLA2G2A |
| GE62645 | NM_005090.1 | PLA2G4B |
| GE54546 | NM_003706.1 | PLA2G4C |
| GE577920 | AB090876.1 | PLA2G4D |
| GE55263 | NM_004253.2 | PLAA |
| GE80967 | NM_000930.2 | PLAT |
| GE59641 | NM_002658.2 | PLAU |
| GE80529 | NM_002659.2 | PLAUR |
| GE58900 | NM_006225.1 | PLCD1 |
| GE86096 | NM_133373.2 | PLCD3 |
| GE83496 | NM_032726.2 | PLCD4 |
| GE81175 | NM_002660.2 | PLCG1 |
| GE57955 | NM_002661.1 | PLCG2 |
| GE54324 | NM_002663.2 | PLD2 |
| GE58954 | NM_000535.2 | PMS2 |
| GE61315 | CD356988.1 | POLB |
| GE60200 | NM_002693.1 | POLG |
| GE54124 | NM_003711.2 | PPAP2A |
| GE86713 | NM_177414.1 | PPAP2B |
| GE54290 | NM_003712.2 | PPAP2C |
| GE56066 | NM_006238.2 | PPARD |
| GE54254 | NM_005037.3 | PPARG |
| GE62667 | NM_013261.2 | PPARGC1A |
| GE59698 | NM_002730.3 | PRKACA |
| GE652786 | AW467479.1 | PRKACB |
| GE577011 | NM_002732.2 | PRKACG |
| GE61216 | M33336.1 | PRKAR1A |
| GE58041 | NM_002735.1 | PRKAR1B |
| GE57918 | NM_002736.2 | PRKAR2B |
| GE86264 | NM_002737.2 | PRKCA |
| GE59687 | NM_002738.5 | PRKCB1 |
| GE57042 | NM_006254.3 | PRKCD |
| GE611116 | AK025126.1 | PRKCE |
| GE57971 | NM_006255.3 | PRKCH |
| GE57727 | L33881.1 | PRKCI |
| GE79759 | NM_006257.2 | PRKCQ |
| GE62614 | NM_006904.6 | PRKDC |
| GE54213 | NM_006017.1 | PROM1 |
| GE57041 | NM_000952.3 | PTAFR |
| GE59570 | NM_000314.2 | PTEN |
| GE79421 | NM_000955.2 | PTGER1 |
| GE58989 | NM_000956.2 | PTGER2 |
| GE80971 | NM_198714.1 | PTGER3 |
| GE57688 | NM_000958.2 | PTGER4 |
| GE55312 | NM_004878.3 | PTGES |
| GE87476 | NM_198938.1 | PTGES2 |
| GE54054 | NM_000959.2 | PTGFR |
| GE53970 | NM_020440.2 | PTGFRN |
| GE57997 | M59979.1 | PTGS1 |
| GE62312 | NM_000963.1 | PTGS2 |
| GE79453 | NM_005607.3 | PTK2 |
| GE59167 | NM_173174.1 | PTK2B |
| GE57124 | NM_006908.3 | RAC1 |
| GE56475 | NM_002878.2 | RAD51L3 |
| GE58929 | NM_002879.2 | RAD52 |
| GE57879 | NM_002890.1 | RASA1 |
| GE81207 | NM_002895.2 | RBL1 |
| GE60009 | NM_005611.2 | RBL2 |
| GE58291 | NM_002899.2 | RBP1 |
| GE57895 | NM_002909.3 | REG1A |
| GE61112 | NM_002908.2 | REL |
| GE61225 | BC033522.1 | RELA |
| GE58104 | NM_006509.2 | RELB |
| GE61154 | X52773.1 | RXRA |
| GE59677 | NM_002964.3 | S100A8 |
| GE80079 | NM_002965.2 | S100A9 |
| GE87242 | NM_133491.2 | SAT2 |
| GE53772 | NM_005063.4 | SCD |
| GE82995 | NM_024906.1 | SCD4 |
| GE58925 | NM_002979.3 | SCP2 |
| GE60412 | NM_002997.4 | SDC1 |
| GE57514 | NM_002998.3 | SDC2 |
| GE56057 | NM_000450.1 | SELE |
| GE57883 | NM_000655.2 | SELL |
| GE58293 | NM_003005.2 | SELP |
| GE62796 | NM_000295.3 | SERPINA1 |
| GE58697 | NM_016186.1 | SERPINA10 |
| GE88319 | BX248259.1 | SERPINA11 |
| GE87109 | NM_173850.2 | SERPINA12 |
| GE60232 | NM_002575.1 | SERPINB2 |
| GE57824 | NM_000602.1 | SERPINE1 |
| GE79528 | NM_006142.3 | SFN |
| GE54530 | NM_003012.3 | SFRP1 |
| GE527352 | NM_003013.2 | SFRP2 |
| GE62558 | NM_003015.2 | SFRP5 |
| GE619667 | NM_000193.2 | SHH |
| GE62087 | NM_021805.1 | SIGIRR |
| GE81228 | NM_003051.2 | SLC16A1 |
| GE57541 | NM_006516.1 | SLC2A1 |
| GE56147 | AI792874.1 | SLC5A12 |
| GE558682 | NM_145913.2 | SLC5A8 |
| GE54526 | NM_004787.1 | SLIT2 |
| GE60408 | NM_003071.2 | SMARCA3 |
| GE54831 | NM_005631.3 | SMO |
| GE59855 | NM_000543.3 | SMPD1 |
| GE58315 | NM_003877.3 | SOCS2 |
| GE79683 | NM_000454.4 | SOD1 |
| GE80932 | NM_000636.1 | SOD2 |
| GE545368 | NM_003118.2 | SPARC |
| GE61335 | NM_000582.2 | SPP1 |
| GE61402 | NM_003150.3 | STAT3 |
| GE83532 | NM_032811.1 | TBRG1 |
| GE57183 | NM_201636.1 | TBXA2R |
| GE58073 | NM_003199.1 | TCF4 |
| GE80031 | NM_003219.1 | TERT |
| GE81248 | NM_003220.1 | TFAP2A |
| GE57647 | NM_003226.2 | TFF3 |
| GE59644 | NM_000660.2 | TGFB1 |
| GE55238 | NM_006022.2 | TGFB1I4 |
| GE81250 | NM_003238.1 | TGFB2 |
| GE59720 | NM_003239.1 | TGFB3 |
| GE61621 | NM_000358.1 | TGFBI |
| GE578892 | NM_003242.3 | TGFBR2 |
| GE80065 | NM_003241.1 | TGM4 |
| GE78973 | S78453.1 | TIMP3 |
| GE81254 | NM_003257.2 | TJP1 |
| GE54434 | NM_003264.2 | TLR2 |
| GE59576 | NM_003266.2 | TLR4 |
| GE54433 | NM_003268.3 | TLR5 |
| GE56816 | NM_016192.2 | TMEFF2 |
| GE59636 | NM_000594.2 | TNF |
| GE79493 | NM_021137.3 | TNFAIP1 |
| GE62903 | NM_003844.2 | TNFRSF10A |
| GE54131 | NM_003842.3 | TNFRSF10B |
| GE54132 | NM_003841.2 | TNFRSF10C |
| GE54225 | AF029761.1 | TNFRSF10D |
| GE562184 | NM_003839.2 | TNFRSF11A |
| GE59899 | NM_000043.3 | TNFRSF6 |
| GE62504 | NM_032945.2 | TNFRSF6B |
| GE59115 | NM_003810.2 | TNFSF10 |
| GE79109 | NM_003701.2 | TNFSF11 |
| GE784965 | NM_000639.1 | TNFSF6 |
| GE55270 | D62608.1 | TOLLIP |
| GE58227 | NM_000546.2 | TP53 |
| GE60333 | NM_003722.3 | TP73L |
| GE79329 | NM_003295.1 | TPT1 |
| GE61891 | NM_003313.2 | TSTA3 |
| GE59639 | NM_001071.1 | TYMS |
| GE840631 | BF516171.1 | TYMS |
| GE495741 | NM_021833.3 | UCP1 |
| GE59585 | NM_003355.2 | UCP2 |
| GE59501 | NM_022803.1 | UCP3 |
| GE57479 | NM_000376.1 | VDR |
| GE59158 | NM_005429.2 | VEGFC |
| GE59707 | NM_007127.1 | VIL1 |
| GE63384 | NM_005430.2 | WNT1 |
| GE83129 | NM_025216.2 | WNT10A |
| GE59476 | NM_003394.2 | WNT10B |
| GE81421 | NM_004626.2 | WNT11 |
| GE59699 | NM_003391.1 | WNT2 |
| GE63181 | NM_024494.1 | WNT2B |
| GE86328 | NM_030753.3 | WNT3 |
| GE507271 | NM_033131.2 | WNT3A |
| GE62675 | NM_030761.3 | WNT4 |
| GE57670 | BQ942339.1 | WNT5A |
| GE61657 | NM_032642.2 | WNT5B |
| GE63133 | W79066.1 | WNT6 |
| GE62528 | NM_004625.3 | WNT7A |
| GE507187 | NM_058238.1 | WNT7B |
| GE897460 | NM_058244.1 | WNT8A |
| GE80654 | NM_003393.2 | WNT8B |
| GE80087 | NM_003395.1 | WNT9A |
| GE543960 | NM_003396.1 | WNT9B |
| GE62786 | NM_000380.2 | XPA |
| GE81853 | NM_003404.3 | YWHAB |
| GE59242 | NM_006761.3 | YWHAE |
| GE53664 | NM_012479.2 | YWHAG |
| GE60439 | NM_003405.2 | YWHAH |
| GE80214 | NM_006826.2 | YWHAQ |
| GE844420 | BF508115.1 | YWHAZ |
| GE81678 | BQ945983.1 | ZBTB33 |

TABLE VI

| Aⱼ^{k} ∩B | k = 1.5 | k = 2 | k = 2.5 | k = 3 |
|---|---|---|---|---|
| j = 0 | 50 | 36 | 23 | 10 |
| j = 1 | 65 | 54 | 35 | 18 |
| j = 2 | 84 | 61 | 46 | 29 |
| j = 3 | 94 | 70 | 51 | 37 |

TABLE VII

| | | Common Genes |
|---|---|---|
| Classification Groups | Sample Size | in A₁² ∩ B |
| (+IR, +Polyps) VS (−IR, −Polyps) at BL1 | 12 | 97 |
| (+IR, +Polyps) on Control VS (+IR, +Polyps) on Legume | 11 | 103 |
| (−IR, −Polyps) on Control VS (−IR, −Polyps) on Legume | 12 | 145 |
| (+IR, +Polyps) on Control VS (−IR, −Polyps) on Control | 11 | 121 |
| (+IR, +Polyps) on Legume VS (−IR, −Polyps) on Legume | 12 | 114 |
| (+IR, +Polyps) VS (−IR, −Polyps) at BL1 & BL2 | 21 | 92 |
| (+Polyps) VS (−Polyps) at BL1 | 23 | 64 |
| (+IR) VS (−IR) at BL1 | 23 | 64 |
| (+Polyps) VS (−Polyps) at BL1 & BL2 | 41 | 59 |
| (+Polyps) on Control VS (+Polyps) on Legume | 21 | 87 |
| (+IR) on Control VS (+IR) on Legume | 23 | 74 |
| (+IR) VS (−IR) at all time points | 86 | 54 |

TABLE VIII

| Gene name | p-value | Fold change |
|---|---|---|
| ALOX12B | 0.1841 | 0.6486 |
| BECN1 | 0.0580 | 0.5140 |
| CDK4 | 0.0370 | 0.5787 |
| DAPK1 | 0.0639 | 1.1258 |
| HOXA3 | 0.0202 | 1.0712 |
| HOXC6 | 0.0134 | 0.4352 |
| ID2 | 0.0626 | 0.9413 |
| IGF1R | 0.0040 | 0.4537 |
| MAPK11 | 0.6291 | 0.7521 |
| NOS3 | 0.0285 | 0.4451 |
| TJP1 | 0.0168 | 0.6092 |
| uCP2 | 0.6330 | 0.7669 |
| WNT1 | 0.7147 | 0.8290 |
| YWHAZ | 0.0298 | 0.4901 |

We claim:

1. A method of detecting a biomarker associated with a colorectal disease or disorder comprising
a) obtaining a fecal sample from a subject exhibiting symptoms associated with or at risk for said colorectal disease or disorder,
b) further isolating at least one biomarker from said fecal sample, and
c) quantifying said biomarker.

2. The method of claim 1, wherein said colorectal disease or disorder is selected from the group consisting of colorectal cancer, colon cancer, large bowel cancer, colonic polyps, anal cancer, general anal and rectal diseases, colitis, Crohn's disease, hemorrhoids, ischemic colitis, ulcerative colitis, diverticulosis, diverticulitis and irritable bowel syndrome.

3. The method of claim 1, wherein said fecal sample is obtained from excretion from said subject.

4. The method of claim 1, wherein said subject is a mammal.

5. The method of claim 1, wherein said biomarker is messenger RNA.

6. The method of claim 1, wherein said biomarker is associated with at least one gene.

7. The method of claim 1, wherein said gene is selected from the group consisting of ACADS, ADAM9, ALOX5, ALOX12B, ATOH1, AXIN2, BAX, BCL, BCL2L12, BECN, CEAL1, CDC42, CSPG2, CSPG4, CXCL-1, EGF, EGFR, F11R, FABP1, FOX, FOXD2, FOXD4L1, FOXL1, FOXL2, FOXP1, FOXP3, FOXD2, FOXO3A, GST-M4, GUCA2A, HMGCL, HOXA1, HOXA11, HOXB2, HOXB3, HOXD10, HSPA12B, ICAM1 (CD54), IGF2, IGFR-1, ITGB4BP, KAI1, KIT, MAPK11, MCM2, MUC5AC, NOX1, NPAT, OGG1, PCNA, PHB, PIK3R1, PIK3C2G, PLCG1, PLCG2, PLCD3, PLCD4, POLG, PRKACB, PTK2B, PTK2, SDC1, SPARC, TGFB2, TGFβ, TGM4, TIMP3, TNF, TNFRSF10B, UCP-3, WNT1, WNT3, Wnt3A, and Wnt5A.

8. A method of measuring biomarkers associated with a colorectal disease or disorder comprising
a) obtaining a first fecal sample from a subject on a first diet;
b) isolating mRNA from said first sample,
c) determining a first mRNA profile;
d) changing the diet of said subject to a second diet;
e) obtaining a second fecal sample from a subject on said second diet;
f) isolating mRNA from said second sample,
g) determining a second mRNA profile; and
h) comparing said first and second mRNA profiles.

9. The method of claim 8, wherein said second mRNA profile indicates a reduced risk for developing adenomas.

10. The method of claim 8, wherein said second diet consists of consuming legumes.

11. The method of claim 8, wherein said first and said second diets have the same energy percentage from dietary fat and dietary protein.

12. The method of claim 11, wherein said energy percentage from dietary fat is at least 30%.

13. The method of claim 11, wherein said energy percentage from dietary protein is at least 15%.

14. The method of claim 8, wherein said change in said diet was after a period of time.

15. The method if claim 11, wherein said period of time is at least one week.
